(19) United States
(12) Patent Application Publication
(10) Pub. No.: US 2010/0282670 A1
(43) Pub. Date: Nov. 11, 2010

(54) METAL RECOVERY DEVICE

(76) Inventor: Eizo NAKAMURA, Tohaku-gun (JP)

Correspondence Address: BIRCH STEWART KOLASCH & BIRCH, PO BOX 747, FALLS CHURCH, VA, 22040-0747, US

(21) Appl. No.: 12/774,350

(22) Filed: May 5, 2010

Related U.S. Application Data
(60) Provisional application No. 61/176,300, filed on May 7, 2009.

(30) Foreign Application Priority Data
Jul. 22, 2009 (JP) P2009-171183

(51) Int. Cl. B01D 39/04; C07K 17/00; C07K 17/14

(52) U.S. Cl. 210/502.1; 530/400; 530/350

(57) ABSTRACT

There is provided a metal recovery device 10 for removing and recovering predetermined metal, from a liquid to be processed, by passing the liquid to be processed through a filter 41, wherein a constituent material of the filter 41 contains a protein that absorbs iron, radium, or heavy metal (except iron).

### BACKGROUND OF THE INVENTION

The present invention relates to a metal recovery device capable of recovering iron, radium or heavy metal (except iron) from a liquid to be processed.

Conventionally, precious metal has frequently been recovered from a liquid to be processed, and made available for reutilization. Although a method for aggregating precious metal by chemical processing has generally been utilized in this recovery, a method for utilizing the property of a protein to absorb precious metal has also been proposed (see Patent Document 1).

Patent Document 1 is a Japanese Unexamined Patent Application Publication No. 2007-185552.

### SUMMARY OF THE INVENTION

However, the above-mentioned recovery method has been capable of recovering precious metal having a relatively low ionization tendency, but has been incapable of sufficiently recovering other metal.

On the other hand, while conducting studies on pleural mesothelioma that is thought to be caused by asbestos, the present inventor discovered a phenomenon in which trace element such as radium is deposited, and found out that this deposition is caused by ferritin known as an iron-binding protein, or hemosiderin which is a partial degradation product of ferritin, for example.

Therefore, the present inventor came up with an idea that metal other than precious metal can also be recovered with high efficiency by utilizing these proteins, and thus achieved the present invention.

The present invention provides a metal recovery device for removing and recovering predetermined metal, from a liquid to be processed, by passing the liquid to be processed through a filter, wherein a constituent material of the filter contains a protein that absorbs iron, radium, or heavy metal (except iron).

In the present invention, iron, radium or heavy metal (except iron) in a liquid to be processed is absorbed into a protein when the liquid to be processed passes through a filter. Iron, radium or heavy metal (except iron) absorbed into the protein can be separated from the protein by known chemical processing, and therefore, the present invention is capable of recovering iron, radium or heavy metal (except iron) from the liquid to be processed.

### BRIEF DESCRIPTION OF THE DRAWINGS

FIG. 1 is a schematic cross-sectional view of a metal recovery device according to one example of the present invention.

FIG. 2 is a partially broken perspective view of a filter plate including a filter according to another example.

FIG. 3 is a schematic cross-sectional view of a metal recovery device according to another example of the present invention.

### DETAILED DESCRIPTION OF THE PREFERRED EMBODIMENTS

A metal recovery device of the present invention removes and recovers predetermined metal, from a liquid to be processed, by passing the liquid to be processed through a filter, and a constituent material of the filter contains a protein that absorbs iron, radium, or heavy metal (except iron).

As the above-mentioned constituent material, a fiber body containing iron, i.e., an iron-containing fiber body, can be used. The iron-containing fiber body is a fiber body made of iron or a fiber body on which iron is supported. The fiber body made of iron can be formed by cutting an iron wire at any length. The fiber body on which iron is supported can be formed by forming an iron layer entirely or partially over a surface of artificial fiber or natural fiber. This iron layer can be formed by a known method such as sputtering, vacuum deposition, electroplating or electroless plating.

As the protein, ferritin, and/or hemosiderin, which is a partial degradation product of ferritin, can be used. This protein has the property of absorbing iron, radium or heavy metal (except iron). This protein is bound to the iron of the iron-containing fiber body. In other words, the iron-containing fiber body serving as the foregoing constituent material contains the protein. In order to allow the protein to bind to the iron of the iron-containing fiber body, the iron-containing fiber body may be immersed in a protein aqueous solution.

In the metal recovery device of the present invention, iron, radium or heavy metal (except iron) in a liquid to be processed is absorbed into a protein when the liquid to be processed passes through a filter. Iron, radium or heavy metal (except iron) absorbed into the protein can be separated from the protein by known chemical processing. Accordingly, the metal recovery device of the present invention is capable of recovering iron, radium or heavy metal (except iron) from the liquid to be processed.

### EXAMPLES

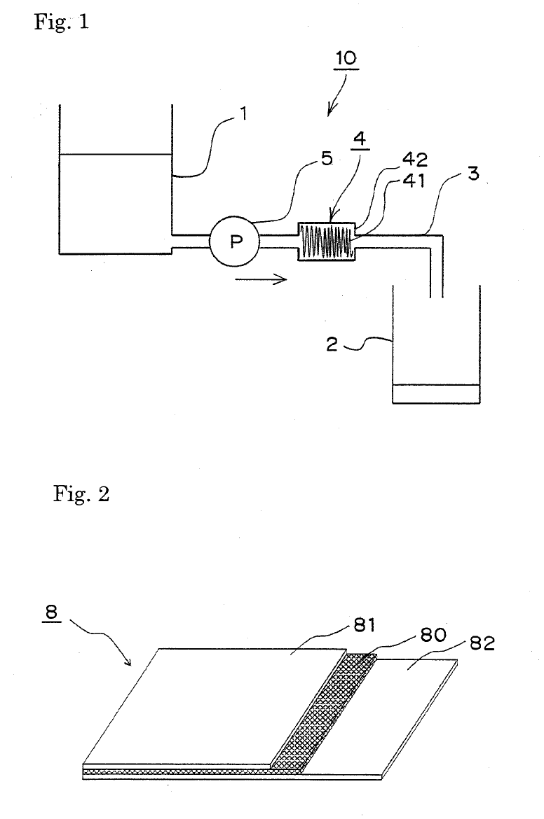

FIG. 1 is a schematic cross-sectional view of a metal recovery device according to the present example. This device 10 includes: a first tank 1 in which a liquid to be processed is stored; a second tank 2 in which a processed liquid is stored; a supply pipe 3 through which both of the tanks 1 and 2 are connected; a filtering part 4 provided at the supply pipe 3; and a supply pump 5 provided at the supply pipe 3 so as to be located upstream of the filtering part 4. The supply pump 5 is an axial-flow pump.

The liquid to be processed is a liquid containing ionized metal or particulate metal, for example.

The first tank 1 may have any form as long as the liquid to be processed can be stored therein.

The liquid to be processed is preferably stored in the first tank 1 after sludge, suspended particles having large grain sizes, and the like have been removed therefrom by using an appropriate filtering device. Thus, occurrence of unnecessary sediment in the first tank 1 can be prevented, and furthermore, degradation in the function of the filtering part 4 due to filtering of sludge, suspended particles and the like can be prevented.

The filtering part 4 includes: a filter 41; and a housing 42 accommodating the filter 41 in an attachable/detachable manner. The filter 41 is in the form of nonwoven fabric, and is filled into the housing 42. A constituent material of the filter 41 is an iron-containing fiber body, and this iron-containing fiber body is an iron fiber body including an iron wire cut at a length of a few millimeters to a few centimeters and having a thickness of a few hundred μm. The filter 41 is made in the following manner. Nonwoven fabric is made by tangling the iron fiber body and a resin fiber body, which is formed of a thermoplastic synthetic resin such as polyester, with each other by a known method, and this nonwoven fabric is immersed in a solution containing ferritin and/or hemosiderin to form a protein layer on a surface of the iron fiber body, thus making the filter 41.

In the metal recovery device 10 having the above-described structure, the liquid to be processed, which is stored in the first tank 1, is supplied to the filtering part 4 at a predetermined pressure upon operation of the supply pump 5. The liquid to be processed, which has been supplied to the filtering part 4, passes through the filter 41. During this time, iron, radium or heavy metal (except iron) in the liquid to be processed is absorbed into the protein of the filter 41. Then, the liquid to be processed, which has passed through the filter 41, will be stored in the second tank 2 as a processed liquid.

Actually, iron, radium or heavy metal (except iron) absorbed into the protein can be separated from the protein by known chemical processing. Accordingly, the metal recovery device 10 having the above-described structure is capable of recovering iron, radium or heavy metal (except iron) from the liquid to be processed.

It should be noted that in the metal recovery device having the above-described structure, the following modifications may be adopted.

(1) As the iron-containing fiber body, a resin fiber body formed of a thermoplastic synthetic resin such as polyester, polyamide, acrylic or the like, on a surface of which an iron layer is formed by a known method, may be used.

(2) The filter 41 may be in the form of woven fabric, a molded body or a mesh body.

(2-1) Woven fabric may be formed by weaving an iron-containing fiber body, or may be formed by twisting together iron-containing fiber bodies to make a twine and by weaving this twine. It should be noted that the twine may be made by twisting together the iron-containing fiber body and “other fiber body”. In such a case, a material for the “other fiber body” is selected in consideration of flexibility, permeability, etc. Furthermore, when a filter is made in the form of the woven fabric, a protein may be bound to the iron-containing fiber body before weaving, or a protein may be bound to the iron-containing fiber body after weaving. In order to allow the protein to bind to the iron-containing fiber body after weaving, the woven fabric may be immersed in a protein aqueous solution.

(2-2) A molded body can be formed by molding an iron-containing fiber body, which is cut at a length of a few millimeters to a few centimeters, into any shape using a known molding method; for example, the molded body can be formed by molding the iron-containing fiber body into a cylindrical shape by a compression molding method. It should be noted that the molded body may be formed by mixing the iron-containing fiber body with “other fiber body”. In such a case, a material for the “other fiber body” is selected in consideration of flexibility, permeability, etc. Furthermore, when a filter is made in the form of the molded body, a protein maybe bound to the iron-containing fiber body before molding, or a protein may be bound to the iron-containing fiber body after molding. In order to allow the protein to bind to the iron-containing fiber body after molding, the molded body may be immersed in a protein aqueous solution.

(2-3) A mesh body can be formed by braiding an iron-containing fiber body into a mesh pattern. It should be noted that the mesh body may be formed by braiding the iron-containing fiber body and “other fiber body” into a mesh pattern. In such a case, a material for the “other fiber body” is selected in consideration of flexibility, permeability, etc. Furthermore, when a filter is made in the form of the mesh body, a protein may be bound to the iron-containing fiber body before braiding, or a protein may be bound to the iron-containing fiber body after braiding. In order to allow the protein to bind to the iron-containing fiber body after braiding, the mesh body may be immersed in a protein aqueous solution.

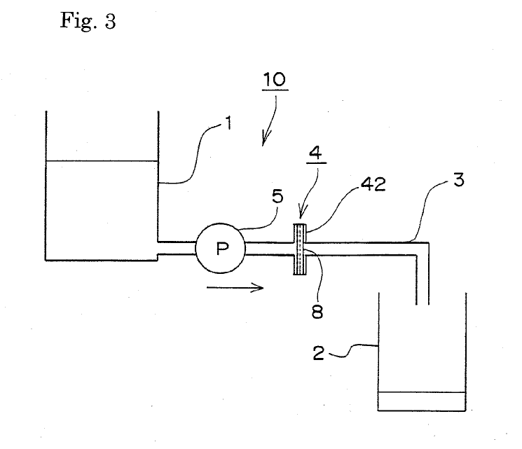

For example, a filter plate 8 including a filter 80, which is made in the form of the mesh body as illustrated in FIG. 2, may be provided at the supply pipe 3 as illustrated in FIG. 3. The filter plate 8 is provided so as to block the supply pipe 3. The filter plate 8 is formed by sandwiching the filter 80 between two sheets of nonwoven fabric 81 and 82 from both sides. Also in the metal recovery device 10 illustrated in FIG. 3, when a liquid to be processed, which has been supplied to the filtering part 4, passes through the filter 80, iron, radium or heavy metal (except iron) in the liquid to be processed is absorbed into the protein of the filter 80. Accordingly, the metal recovery device 10 illustrated in FIG. 3 is also capable of recovering iron, radium or heavy metal (except iron) from the liquid to be processed.

(3) Instead of the iron-containing fiber body, iron-containing amphibole asbestos, e.g., amosite and/or crocidolite, can be used. However, in terms of environmental protection, the use of asbestos should be avoided.

A metal recovery device of the present invention is capable of recovering iron, radium or heavy metal (except iron) from a liquid to be processed, and is thus industrially very useful.

What is claimed is:

1. A metal recovery device for removing and recovering predetermined metal, from a liquid to be processed, by passing the liquid to be processed through a filter,
wherein a constituent material of the filter contains a protein that absorbs iron, radium, or heavy metal (except iron).

2. The metal recovery device according to claim 1,
wherein the constituent material is an iron-containing fiber body, and
wherein the protein is bound to iron of the iron-containing fiber body.

3. The metal recovery device according to claim 2,
wherein the iron-containing fiber body is a fiber body made of iron or a fiber body on which iron is supported.

4. The metal recovery device according to claim 2,
wherein the protein is ferritin or hemosiderin.
